(19) United States
(12) Patent Application Publication
(10) Pub. No.: US 2024/0099668 A1
(43) Pub. Date: Mar. 28, 2024

(54) MONITORING METHOD AND MONITOR

(71) Applicant: NIHON KOHDEN CORPORATION, Tokyo (JP)

(72) Inventors: Rie MUNESHIMA, Tokorozawa-shi, Saitama (JP); Hiroko HAGIWARA, Tokorozawa-shi, Saitama (JP)

(73) Assignee: NIHON KOHDEN CORPORATION, Tokyo (JP)

(21) Appl. No.: 18/273,460

(22) PCT Filed: Feb. 9, 2022
(86) PCT No.: PCT/JP2022/005214

§ 371 Date: Jul. 20, 2023

(30) Foreign Application Priority Data
Feb. 22, 2021 (JP) 2021-025949

(51) Int. Cl. A61B 5/00

(52) U.S. Cl. CPC A61B 5/746; A61B 5/7221

(57) ABSTRACT

A monitoring method includes a calculation step and an output step. The calculation step includes calculating an alarm generation time, in which at least one of a vital alarm notifying an abnormality of physiological information and a technical alarm is generated, or a ratio of the alarm generation time. The output step includes outputting the calculated alarm generation time or the calculated ratio.

### TECHNICAL FIELD

The presently disclosed subject matter relates to a monitoring method, a monitor, and a program.

### BACKGROUND

In a patient monitor that displays physiological information of a patient and the like, a technique of outputting a vital alarm for notifying an abnormality when an abnormality is detected in the physiological information or a technical alarm for notifying an abnormality of a device or a system that measures the physiological information is known.

In relation to such a technique, the following related art is disclosed in the following Patent Literature 1. A physiological information waveform is displayed in a first region of a screen, and vital data obtained by arranging generation history information of vital alarms for each living body in time series and converting the generation history information into display data and technical data obtained by arranging generation history information of technical alarms for each living body in time series and converting the generation history information into display data are displayed in a second region. Accordingly, it is possible to clarify the alarm generation history.

### CITATION LIST

### Patent Literature

Patent Literature 1: Japanese Patent No. 5110270

### SUMMARY

### Technical Problem

However, when the vital alarm or the technical alarm is notified during physiological information measurement, a medical worker needs to go to a place of a patient and the like to take action in order to confirm a state of the patient and the like and a state of a sensor and the like that detects physiological information. The related art described above does not meet the desire to quantitatively grasp a workload of the medical worker caused by responding to the generation of an alarm.

The presently disclosed subject matter has been made to solve the above-described problems. That is, it is an object of the presently disclosed subject matter to provide a monitoring method, a monitor, and a program capable of quantitatively grasping a workload of a medical worker caused by generation of an alarm during physiological information measurement.

### Solution to Problem

(1) A monitoring method includes: a calculation step of calculating an alarm generation time, in which at least one of a vital alarm notifying an abnormality of physiological information and a technical alarm is generated, or a ratio of the alarm generation time; and an output step of outputting the calculated alarm generation time or the calculated ratio.

(2) A monitor includes: a calculation unit configured to calculate an alarm generation time, in which at least one of a vital alarm notifying an abnormality of physiological information and a technical alarm is generated, or a ratio of the alarm generation time; and an output unit configured to output the calculated alarm generation time or the calculated ratio.

(3) A program for causing a computer to execute the following steps: a calculation step of calculating an alarm generation time, in which at least one of a vital alarm notifying an abnormality of physiological information and a technical alarm is generated, or a ratio of the alarm generation time; and an output step of outputting the calculated alarm generation time or the calculated ratio.

### Effects of the Invention

A time during which at least one of a vital alarm notifying an abnormality of physiological information and a technical alarm is generated or a ratio of the time is calculated and output. Accordingly, it is possible to quantitatively grasp a workload of a medical worker caused by generation of an alarm during physiological information measurement.

### BRIEF DESCRIPTION OF DRAWINGS

FIG. 1 is a schematic configuration diagram of a medical system.

FIG. 2 is a block diagram of a hardware configuration of a bedside monitor.

FIG. 3 is a block diagram of a hardware configuration of a transmitter.

FIG. 4 is a block diagram of a hardware configuration of a centralized receiver.

FIG. 5 is a block diagram of a hardware configuration of a central monitor.

FIG. 6 illustrates an alarm generation time, a ratio of the alarm generation time, and the like for each time zone.

FIG. 7 illustrates a graph of the ratio of the alarm generation time for each time zone.

FIG. 8 illustrates the alarm generation time, the ratio of the alarm generation time, and the like for each time zone in a distinguishable manner for each type of an alarm.

FIG. 9 illustrates a graph in which the ratio of the alarm generation time for each time zone is indicated in a distinguishable manner for each type of an alarm.

FIG. 10 illustrates an alarm generation time, a ratio of the alarm generation time, and the like for each day.

FIG. 11 illustrates a bar graph of the ratio of the alarm generation time for each day.

FIG. 12 illustrates a pie chart of the ratio of the alarm generation time for each day.

FIG. 13 illustrates the ratio of the alarm generation time for each time zone for each predetermined attribute of an alarm.

FIG. 14 illustrates a bar graph in which the ratio of the alarm generation time for each time zone is indicated in a distinguishable manner for each predetermined attribute of an alarm.

FIG. 15 illustrates an alarm generation time and an alarm non-generation time for each time zone.

FIG. 16 illustrates a bar graph of the alarm generation time and the alarm non-generation time for each time zone.

FIG. 17 is a flowchart illustrating an operation of the central monitor.

### DESCRIPTION OF EMBODIMENTS

Hereinafter, a monitoring method, a monitor, and a program according to an embodiment of the presently disclosed subject matter will be described in detail with reference to the drawings. In the drawings, the same elements are denoted by the same reference numerals, and a repetitive description thereof will be omitted.

### <Medical System 10>

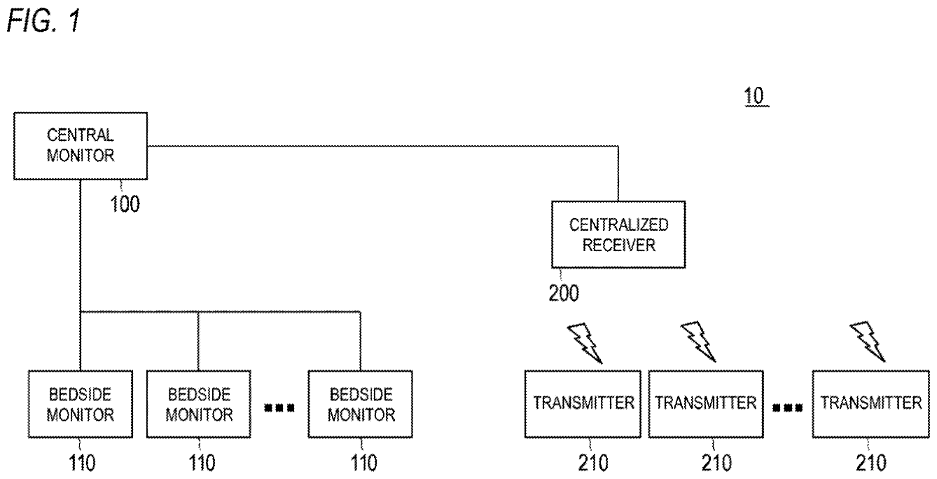

FIG. 1 is a schematic configuration diagram of a medical system 10. The medical system 10 includes a central monitor 100, bedside monitors 110, a centralized receiver 200, and transmitters 210. One or more bedside monitors 110 and one or more transmitters 210 may be provided. Only one of the bedside monitor 110 and the transmitter 210 may be provided. The central monitor 100 constitutes a monitor.

The central monitor 100 and the bedside monitor 110 are communicably connected to each other via a wired or wireless network. The network is, for example, a local area network (LAN), a wide area network (WAN), and the like. As a communication standard of the network, for example, Ethernet (registered trademark), Wi-Fi (registered trademark), Bluetooth (registered trademark), or 5G may be used.

The central monitor 100 and the centralized receiver 200 are communicably connected to each other via a wired or wireless network. The network is, for example, a LAN, a WAN, and the like, furthermore, for example, Ethernet (registered trademark), Wi-Fi (registered trademark), Bluetooth (registered trademark), or 5G may be used as a communication standard of the network.

The centralized receiver 200 and the transmitter 210 are communicably connected to each other via a wireless network. The network is, for example, a wireless LAN and the like, furthermore, for example, Wi-Fi (registered trademark), Bluetooth (registered trademark), or 56 may be used as a communication standard of the network.

### <Bedside Monitor 110>

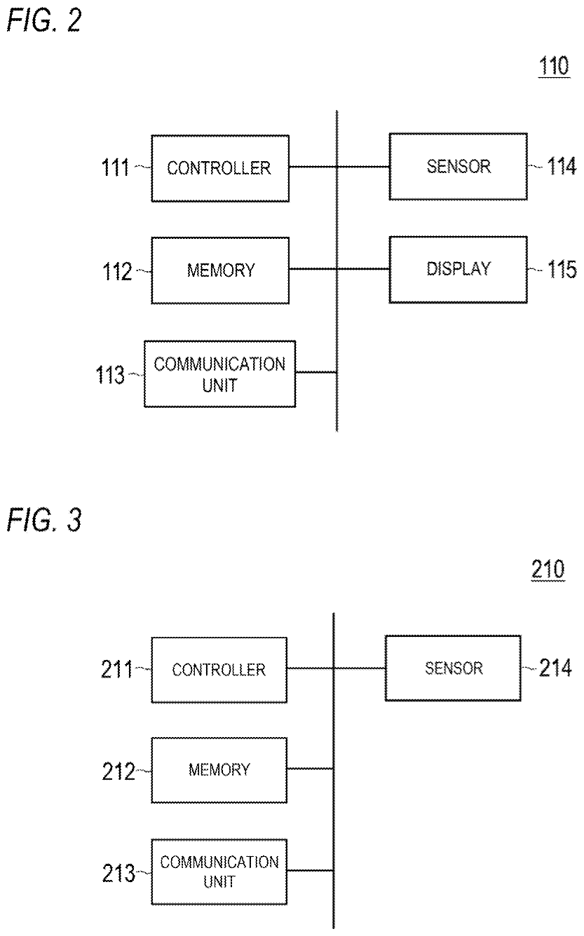

FIG. 2 is a block diagram of a hardware configuration of the bedside monitor 110. The bedside monitor 110 includes a controller 111, one or more memory 112, a communication unit 113, a sensor 114, and a display 115. These components are connected to each other by a bus. Some of the components may be connected to the bus by wireless communication. The bedside monitor 110 may be provided, for example, for each bed of a patient or for each room of a patient.

The controller 111 includes, for example, a central processing unit (CPU) and a random access memory (RAM), and controls each component of the bedside monitor 110 and performs various calculations.

The controller 111 transmits physiological information detected by the sensor 114 to the central monitor 100 by the communication unit 113. The controller 111 detects an abnormality in the physiological information, and when the abnormality is detected, the controller 111 transmits a vital alarm for notifying the abnormality in the physiological information and the physiological information to the central monitor 100 by the communication unit 113. The physiological information includes, for example, an electrocardiogram (ECG), an arterial oxygen saturation (SpO2), an invasive blood pressure (IBP), a non-invasive blood pressure (NIBP), respiration (RESP), and carbon dioxide (CO₂) of exhalation and inspiration. The abnormality of the physiological information can be detected by exceeding an upper limit value, a lower limit value, or an upper and lower limit value set for the physiological information.

The controller 111 detects an abnormality of a measurement device, a system, and the like, and when the abnormality is detected, the controller 111 transmits a technical alarm notifying the abnormality to the central monitor 100 by the communication unit 113. The abnormality notified by the technical alarm includes, for example, an abnormality of a device and the like including a measurement device (a device or an element) constituting the sensor 114, an abnormality of a mounting state such as detachment of the sensor 114 mounted on a patient, an abnormality of a measurement environment such as radio wave interruption or noise mixing, and the like. Specifically, examples of the technical alarm include “electrode confirmation”, “radio wave interruption”, and “probe confirmation”. The “electrode confirmation” is an alarm generated due to detachment of an electrode lead from an ECG electrode attached to the patient, floating of the ECG electrode from the body, disconnection of the electrode lead, and the like. The “radio wave interruption” is an alarm indicating that a reception state of radio waves is bad, interference occurs, information cannot be received, and the like. The “probe confirmation” is an alarm generated due to a SpO2 probe-off attached to a patient from the body, the SpO2 probe-off from a relay code or a measurement device, disconnection or short circuit of the SpO2 probe, and the like.

The vital alarm and the technical alarm may be continuously transmitted to the central monitor 100 while an abnormality is detected.

The controller 111 can transmit the physiological information and the alarm in association with identification information for identifying the subject (patient and the like) of the physiological information to the central monitor 100 together with the identification information. The identification information includes, for example, a bed number of a patient, an ID of the patient, an IP address of the bedside monitor 110, and the like. Information such as a measurement date and time may be added to the transmitted physiological information.

The memory 112 is implemented by, for example, a solid state drive (SSD), and stores various data.

The communication unit 113 is an interface for communicably connecting the bedside monitor 110 and the central monitor 100. The communication unit 113 may include, for example, an input terminal, an antenna, a front end circuit, and the like.

The sensor 114 is a device or an element that detects the physiological information. The sensor 114 includes, for example, an electrocardiogram measurement electrode, a SpO2 probe, and the like.

The display 115 displays (outputs) the physiological information, an alarm generation time or a ratio of the alarm generation time described later in a visually recognizable manner. The display 115 may be implemented by, for example, a liquid crystal display and the like.

### <Transmitter 210>

FIG. 3 is a block diagram of a hardware configuration of the transmitter 210. The transmitter 210 is a device that is carried by a patient, detects physiological information of the patient, and wirelessly transmits the detected physiological signal to the centralized receiver 200. The transmitter 210 includes a controller 211, one or more memory 212, a communication unit 213, and a sensor 214. The basic configurations of the controller 211, the memory 212, and the sensor 214 of the transmitter 210 are the same or similar as the basic configurations of the corresponding components of the bedside monitor 110, and thus a repetitive description thereof will be omitted.

The controller 211 wirelessly transmits the physiological information detected by the sensor 214 to the central monitor 100 by the communication unit 213.

The communication unit 213 is an interface for communicably connecting the transmitter 210 and the central monitor 100 via a wireless network and the like. The communication unit 213 may include, for example, an input terminal, an antenna, a front end circuit, and the like. The transmitter 210 and the central monitor 100 are connected to each other via a network such as a wireless LAN.

The transmitter 210 may further include a display (not illustrated) that displays the physiological information in a visually recognizable manner.

### <Centralized Receiver 200>

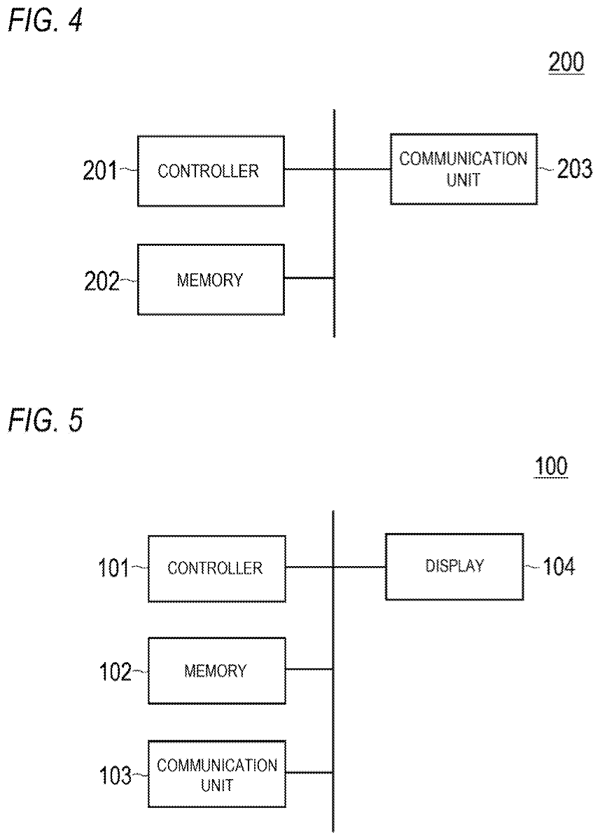

FIG. 4 is a block diagram of a hardware configuration of the centralized receiver 200. The centralized receiver 200 includes a controller 201, one or more memory 202, and a communication unit 203. These components are connected to each other by a bus. Since the basic configurations of the controller 201, the memory 202, and the communication unit 203 of the centralized receiver 200 are the same or similar as the basic configurations of the corresponding components of the bedside monitor 110 and the transmitter 210, a repetitive description will be omitted.

The controller 201 receives the physiological information from each transmitter 210 by the communication unit 203. The controller 201 detects an abnormality in the physiological information, and when the abnormality is detected, the controller 201 transmits a vital alarm and the physiological information to the central monitor 100 by communication unit 203.

The controller 201 detects an abnormality of a measurement device, a system, and the like, and when the abnormality is detected, the controller 201 transmits a technical alarm to the central monitor 100 by the communication unit 203.

The vital alarm and the technical alarm may be continuously transmitted to the central monitor 100 while an abnormality is detected.

When the controller 201 receives the above-described identification information from the transmitter 210, the controller 201 may transmit physiological information and an alarm in association with the identification information to the central monitor 100 together with the identification information. The identification information includes, for example, a bed number of a patient, an ID of the patient, an IP address of the transmitter 210, and the like.

The communication unit 203 is an interface for communicably connecting the centralized receiver 200 to the central monitor 100 and the transmitter 210. The communication unit 203 may include, for example, an input terminal, an antenna, a front end circuit, and the like.

### <Central Monitor 100>

FIG. 5 is a block diagram of a hardware configuration of the central monitor 100. The central monitor 100 includes a controller 101, one or more memory 102, a communication unit 103, and a display 104. These components are connected to each other by a bus. Since the basic configurations of these components are the same or similar as the basic configurations of the corresponding components of the bedside monitor 110, a repetitive description thereof will be omitted.

The controller 101 receives physiological information, a vital alarm, and a technical alarm from each of the bedside monitors 110 and each of the transmitters 210 by the communication unit 103.

The memory 102 stores the physiological information, the vital alarm, and the technical alarm in association with the received time. The physiological information, the vital alarm, and the technical alarm may be stored in association with the identification information.

The controller 101 calculates an alarm generation time. The alarm generation time is a time in which at least one of a vital alarm and a technical alarm is generated. Hereinafter, when the vital alarm and the technical alarm are not distinguished from each other, these alarms are also simply referred to as alarms. As described above, the same alarm can be continuously transmitted. In this case, the alarm generation time may be a time from when the alarm is received to when the alarm is no longer received. The controller 101 calculates a ratio of the alarm generation time. The ratio of the alarm generation time to a predetermined time is a ratio of the alarm generation time to the predetermined time. The predetermined time may be, for example, a work zone, one hour, one day, one week, one month, six months, or one year.

The controller 101 may calculate and output at least one of the alarm generation time and the ratio of the alarm generation time for each continuous time zone. The output includes a case of displaying on the display 104, a case of transmitting to another device by the communication unit 103, and the like.

The controller 101 can output at least one of the alarm generation time and the ratio of the alarm generation time in a distinguishable manner for each type of an alarm (the vital alarm and the technical alarm).

The controller 101 may calculate the alarm generation time (a sum of the alarm generation times) or the ratio of the alarm generation time for each group to which the subject of the physiological information belongs based on the identification information, and output the alarm generation time or the ratio of the alarm generation time calculated for the group designated by a user in the input unit (not illustrated) such as a touch panel of the central monitor 100.

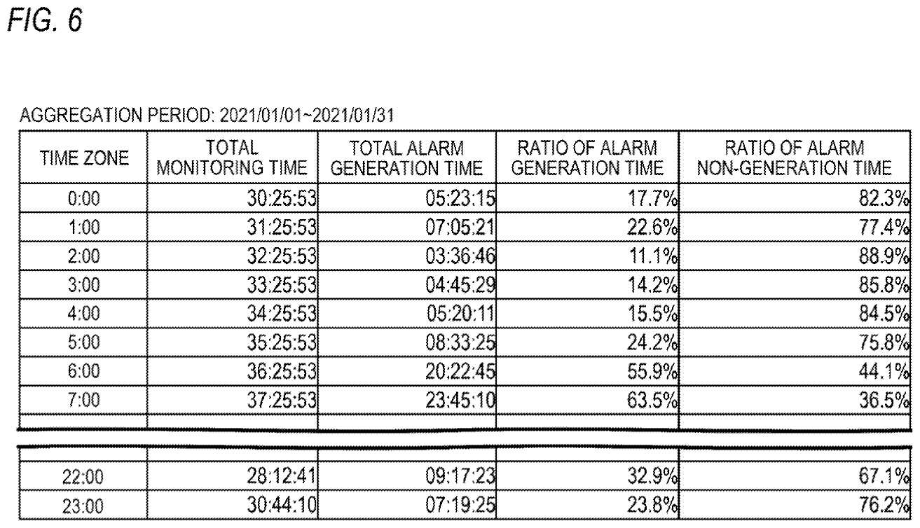

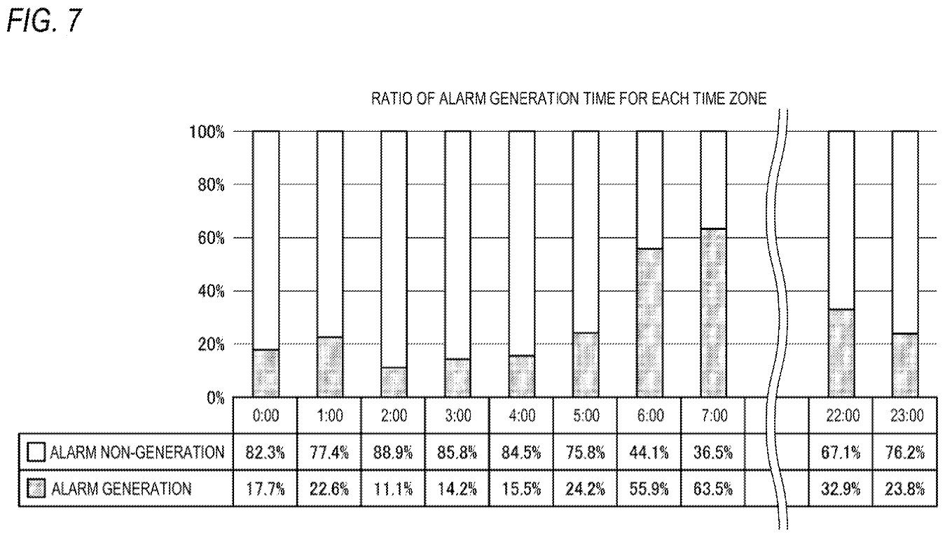

FIG. 6 illustrates the alarm generation time, the ratio of the alarm generation time, and the like for each tune zone. FIG. 7 illustrates a graph of the ratio of the alarm generation time for each time zone.

In the example of FIG. 6, an aggregation result of an aggregation period for one month of a total monitoring time, a total alarm generation time, the ratio of the alarm generation time, and a ratio of an alarm non-generation time are illustrated for each time zone. An average number of subjects to be monitored is 13. The subjects to be aggregated are patients in the same ward, which is a predetermined group. The total monitoring time is a sum of the times during which the physiological signal is monitored in each time zone, and is calculated based on an entering and leaving time of each bed. The total alarm generation time is the sum of the alarm generation times during the total monitoring time. The ratio of the alarm generation time is a ratio of the total alarm generation time to the total monitoring time (total alarm generation time/total monitoring time×100). A total alarm non-generation time is a sum of times other than the alarm generation time in the total monitoring time. The ratio of the alarm non-generation time is a ratio of the total alarm non-generation time to the total monitoring time (total alarm non-generation time/total monitoring time×100).

As described above, the predetermined group to be aggregated may be the group designated by the user. The subject to be subjected to the aggregation of the physiological information may be a patient and the like who is in charge of a care staff belonging to the same work time zone.

The aggregation period can be optionally set, and may be a work zone, one hour, one day, one week, one month, six months, or one year. Further, the monitoring target is optional, and may be one person.

In the example of FIG. 7, the ratio of the alarm generation time is indicated by a bar graph for each time zone.

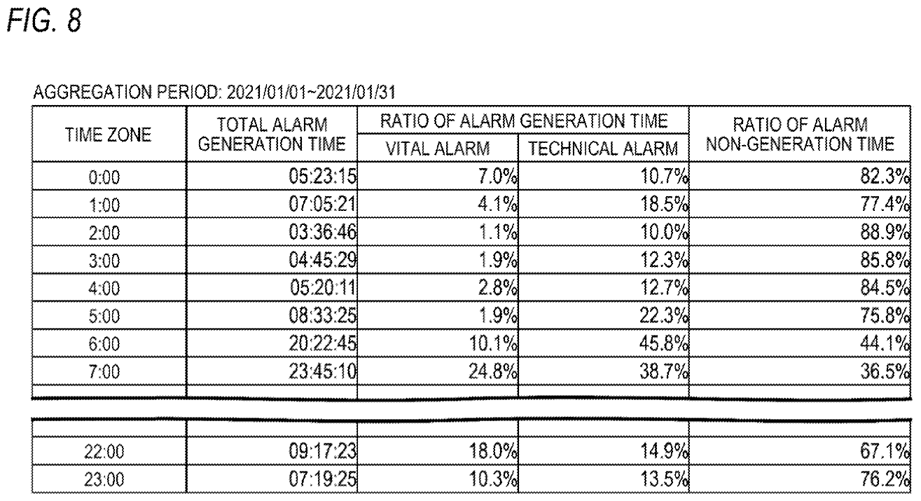

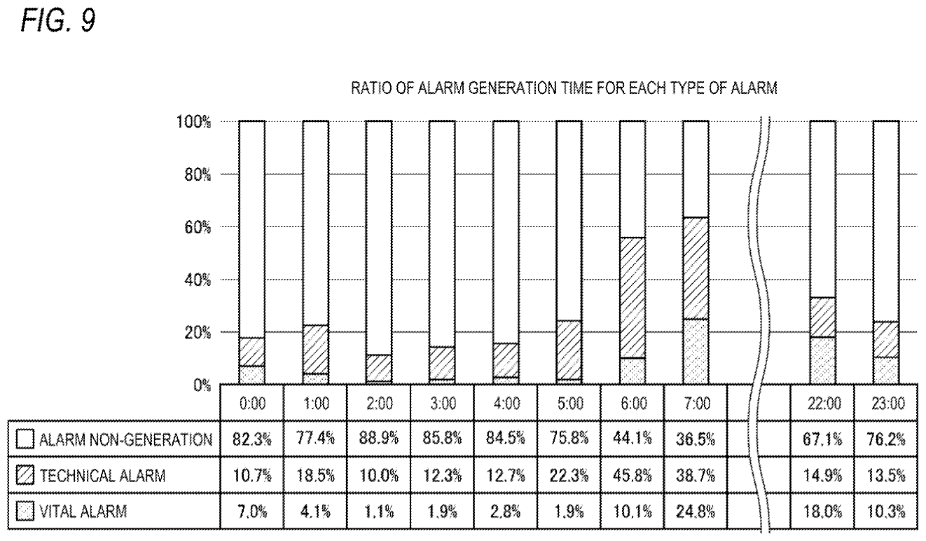

FIG. 8 illustrates the alarm generation time, the ratio of the alarm generation time, and the like for each time zone in a distinguishable manner for each type of an alarm. FIG. 9 illustrates a graph in which the ratio of the alarm generation time for each time zone is indicated in a distinguishable manner for each type of an alarm.

In the example of FIG. 8, a ratio of a generation time of the vital alarm and a ratio of a generation time of the technical alarm are displayed in different rows so as to be distinguishable from each other. That is, the ratio of the alarm generation time is displayed in a distinguishable manner for each type of an alarm.

In the example of FIG. 9, the ratio of the generation time of the vital alarm and the ratio of the generation time of the technical alarm are displayed as bar graphs divided by different colors or patterns so as to be distinguishable from each other.

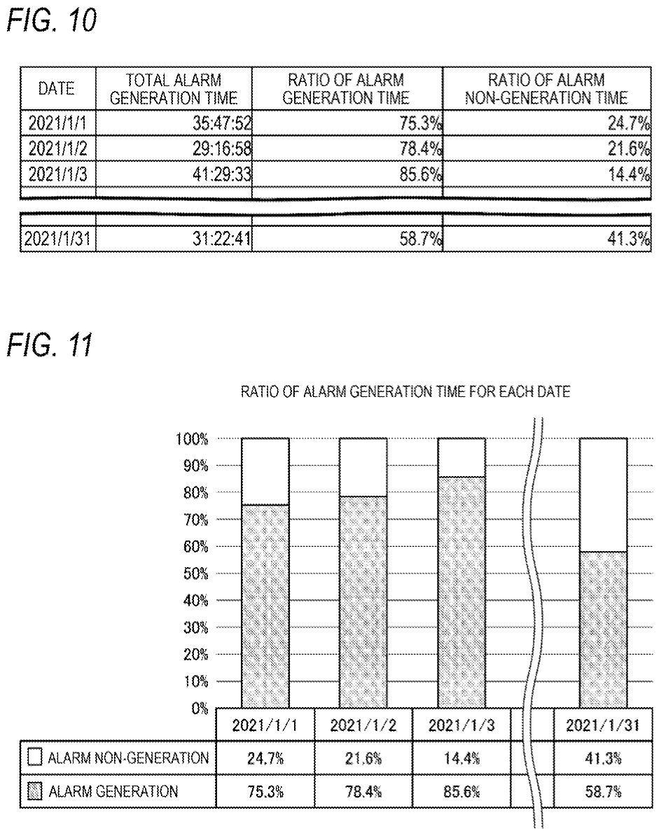

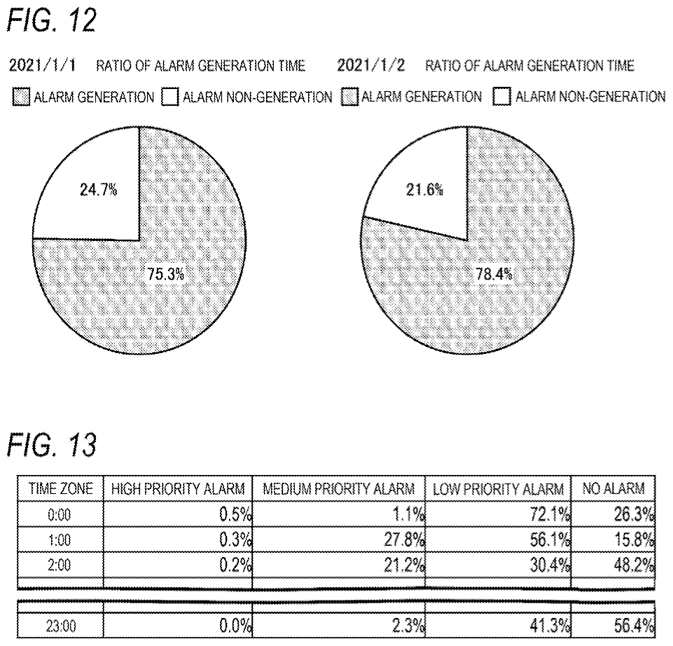

FIG. 10 illustrates an alarm generation time, a ratio of the alarm generation time, and the like for each day. FIG. 11 illustrates a bar graph of the ratio of the alarm generation time for each day. FIG. 12 illustrates a pie chart of the ratio of the alarm generation time for each day.

In the example of FIG. 10, the aggregation result of the aggregation period for one month of the total alarm generation time, the ratio of the alarm generation time, and the ratio of the alarm non-generation time are illustrated for each day (date). In the example of FIG. 11, the ratio of the alarm generation time and the like are indicated by a bar graph for each day. In the example of FIG. 12, the ratio of the alarm generation time and the like are indicated by a pie chart for each day.

The alarm generation time and the ratio of the alarm generation time may be displayed in another form such as a line graph.

The controller 101 may calculate the alarm generation time or the ratio of the alarm generation time for each predetermined attribute of the alarm, and output the alarm generation time or the ratio of the alarm generation time in a distinguishable manner for each attribute. The predetermined attribute includes, for example, a high priority, a medium priority, and a low priority that are set according to the degree of emergency or the degree of importance. The predetermined attribute may be a detailed parameter of the vital alarm or the technical alarm, or may be a work zone of a care staff and the like. The detailed parameter of the vital alarm includes, for example, a decrease in arterial oxygen saturation and a decrease in blood pressure. The detailed parameter of the technical alarm includes, for example, “electrode confirmation”, “radio wave interruption”, and “probe confirmation”.

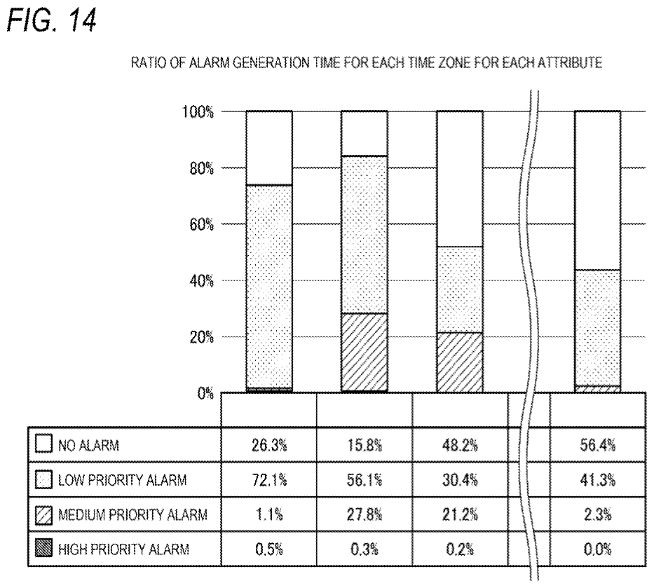

FIG. 13 illustrates the ratio of the alarm generation time for each time zone for each predetermined attribute of an alarm. FIG. 14 illustrates a bar graph in which the ratio of the alarm generation time for each time zone is indicated in a distinguishable manner for each predetermined attribute of an alarm.

In the example of FIG. 13, the ratio of the alarm generation time is illustrated such that a high priority alarm, a medium priority alarm, and a low priority alarm can be distinguished for each continuous time zone. In the example of FIG. 14, the ratio of the alarm generation time is displayed as a bar graph in which attributes of the high priority, the medium priority, and the low priority are classified by different colors or patterns for each continuous time zone, such that the ratios of the alarm generation times are distinguishable from each other.

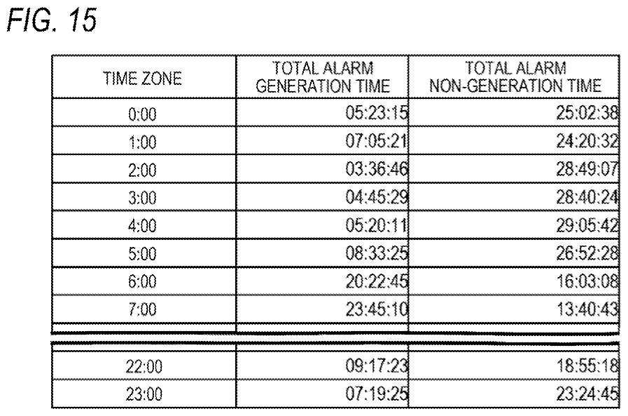

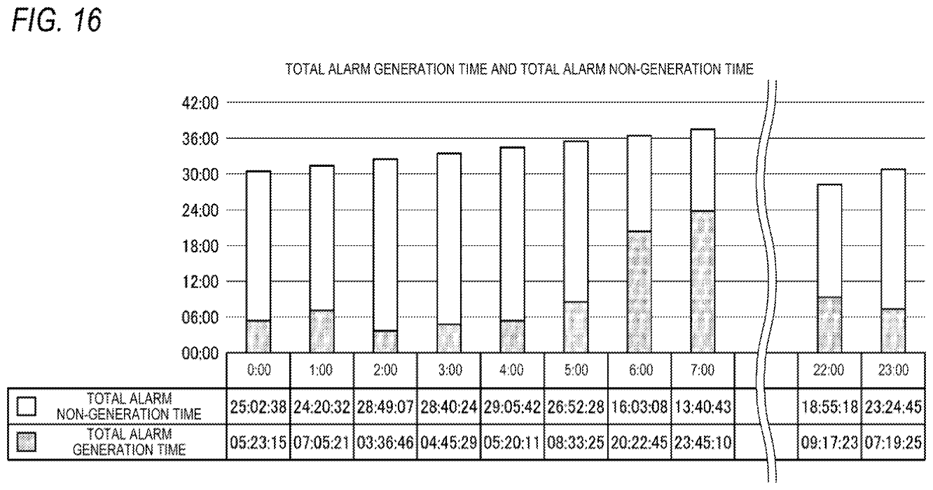

FIG. 15 illustrates an alarm generation time and an alarm non-generation time for each time zone. FIG. 16 illustrates a bar graph of the alarm generation time and the alarm non-generation time for each time zone.

In the example of FIG. 15, an aggregation result for one month of the total alarm generation time (the sum of the alarm generation times) and the total alarm non-generation time (the sum of the alarm non-generation times) is illustrated for each time zone. In the example of FIG. 16, an aggregation result for one month of the total alarm generation time and the total alarm non-generation time is illustrated as a bar graph for each time zone.

An operation of the central monitor 100 will be described.

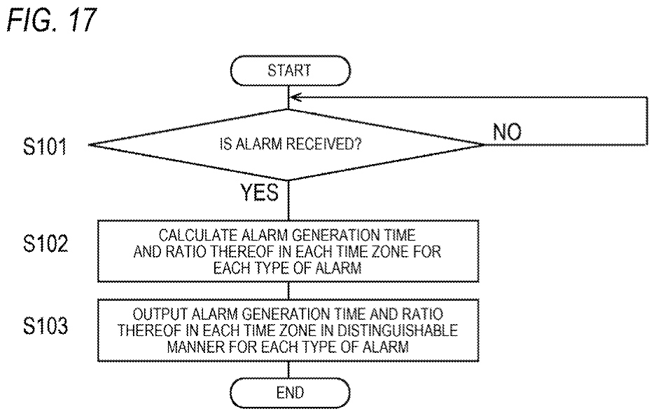

FIG. 17 is a flowchart illustrating the operation of the central monitor 100. The flowchart can be executed by the controller 101 of the central monitor 100 in accordance with a program. Incidentally, a non-transitory computer-readable medium in which the program for executing the processing according to the present embodiment has been stored may be used.

The controller 101 determines whether an alarm has been received (S101). When the controller 101 determines that the alarm is not received (S101: NO), the controller 101 repeatedly executes step S101.

When the controller 101 determines that the alarm has been received (S101: YES), the controller 101 calculates (measures) a time during which the alarm is received, thereby calculating the alarm generation time and the ratio of the alarm generation time in each time zone for each type of an alarm (S102).

The controller 101 outputs the alarm generation time and the ratio of the alarm generation time in each time zone in a distinguishable manner for each type of an alarm (S103).

The present embodiment has the following effects.

An alarm generation time, in which at least one of a vital alarm notifying an abnormality of physiological information and a technical alarm notifying an abnormality of a measurement device and the like is generated, or a ratio of the alarm generation time is calculated and output. Accordingly, it is possible to quantitatively grasp a workload of a medical worker caused by generation of an alarm during physiological information measurement.

Further, at least one of the vital alarm and the technical alarm is received, and the alarm generation time or the ratio of the alarm generation time is calculated based on at least one of the received vital alarm and the received technical alarm. Accordingly, it is possible to grasp the workload of the medical worker caused by the generation of the alarm in a wide range.

Further, the alarm generation time or the ratio of the alarm generation time is output in a distinguishable manner for each type of an alarm. Accordingly, it is possible to quantitatively grasp the workload of the medical worker for each type of an alarm.

Further, the alarm generation time or the ratio of the alarm generation time for each continuous time zone is calculated, and the alarm generation time or the ratio of the alarm generation time for each time zone is output. Accordingly, it is possible to quantitatively grasp the workload of the medical worker for each time zone.

Further, the alarm generation time or the ratio of the alarm generation time for each predetermined attribute of the alarm is calculated, and the alarm generation time or the ratio of the alarm generation time is output in a distinguishable manner for each attribute. Accordingly, it is possible to more accurately grasp the workload of the medical worker.

Further, each of the vital alarm and the technical alarm is associated with identification information for identifying a subject of the physiological information. A sum of the alarm generation times in which at least one of the vital alarm and the technical alarm is generated or a ratio of the sum for each group to which the subject belongs is calculated based on the identification information. The sum of the alarm generation times or the ratio of the sum calculated for the designated group is output. Accordingly, it is possible to accurately grasp the workload for the group for which the workload is desired to be grasped.

Further, the technical alarm is at least one of an alarm notifying an abnormality of a measurement device of the physiological information, an abnormality of a mounting state of a sensor to a subject, and an abnormality of a measurement environment of the physiological information. Accordingly, it is possible to easily and efficiently grasp the workload of the medical worker and the like by the alarm.

Although the embodiment of the presently disclosed subject matter have been described in detail above, the presently disclosed subject matter is not limited to the above-described embodiment.

For example, some or all of the functions implemented by the program in the above-described embodiment may be implemented by hardware such as a circuit.

Further, in the above-described flowchart, some steps may be omitted, or other steps may be added. Further, some of the steps may be executed at the same time, or one step may be divided into a plurality of steps and executed.

This application claims priority to Japanese Patent Application No. 2021-025949 filed on Feb. 22, 2021, the entire content of which is incorporated herein by reference.

### INDUSTRIAL APPLICABILITY

According to the presently disclosed subject matter, it is possible to provide a monitoring method, a monitor, and a program capable of quantitatively grasping a workload of a medical worker caused by generation of an alarm during physiological information measurement.

1. A monitoring method, comprising:
a calculation step of calculating an alarm generation time, in which at least one of a vital alarm notifying an abnormality of physiological information and a technical alarm is generated, or a ratio of the alarm generation time; and
an output step of outputting the calculated alarm generation time or the calculated ratio.

2. The monitoring method according to claim 1, further comprising:
a reception step of receiving at least one of the vital alarm and the technical alarm, wherein
the calculation step calculates the alarm generation time or the ratio based on at least one of the received vital alarm and the received technical alarm.

3. The monitoring method according to claim 1, wherein
the output step outputs the alarm generation time or the ratio in a distinguishable manner for a type of the alarm.

4. The monitoring method according to claim 1, wherein
the calculation step calculates the alarm generation time or the ratio for each continuous time zone, and
the output step outputs the alarm generation time or the ratio for each time zone.

5. The monitoring method according to claim 1, wherein
the calculation step calculates the alarm generation time or the ratio for each predetermined attribute of the alarm, and
the output step outputs the alarm generation time or the ratio in a distinguishable manner for each attribute.

6. The monitoring method according to claim 1, wherein
each of the vital alarm and the technical alarm is associated with identification information for identifying a subject of the physiological information,
the calculation step calculates, based on the identification information, a sum of the alarm generation times in which at least one of the vital alarm and the technical alarm is generated or a ratio of the sum for each group to which the subject belongs or for each subject, and
the output step outputs the sum or the ratio of the sum calculated for a designated group.

7. The monitoring method according to claim 1, wherein
the technical alarm is at least one of an alarm notifying an abnormality of a measurement device of the physiological information, an abnormality of a mounting state of a sensor to a subject, and
an abnormality of a measurement environment of the physiological information.

8. A monitor comprising:
a calculation unit configured to calculate an alarm generation time, in which at least one of a vital alarm notifying an abnormality of physiological information measured by a sensor and a technical alarm is generated, or a ratio of the alarm generation time; and
an output unit configured to output the calculated alarm generation time or the calculated ratio.

9. The monitor according to claim 8, further comprising:
a first communication unit configured to receive at least one of the vital alarm and the technical alarm, wherein
the calculation unit calculates the alarm generation time or the ratio based on at least one of the received vital alarm and the received technical alarm.

10. The monitor according to claim 8, wherein
the output unit outputs the alarm generation time or the ratio in a distinguishable manner for a type of the alarm.

11. The monitor according to claim 8, wherein
the calculation unit calculates the alarm generation time or the ratio for each continuous time zone, and
the output unit outputs the alarm generation time or the ratio for each time zone.

12. The monitor according to claim 8, wherein
the calculation unit calculates the alarm generation time or the ratio for each predetermined attribute of the alarm, and
the output unit outputs the alarm generation time or the ratio in a distinguishable manner for each attribute.

13. The monitor according to claim 8, wherein
each of the vital alarm and the technical alarm is associated with identification information for identifying a subject of the physiological information,
the calculation unit calculates, based on the identification information, a sum of the alarm generation times in which at least one of the vital alarm and the technical alarm is generated or a ratio of the sum for each group to which the subject belongs or for each subject, and
the output unit outputs the sum or the ratio of the sum calculated for a designated group.

14. The monitor according to claim 8, wherein
the technical alarm is at least one of an alarm notifying an abnormality of a measurement device of the physiological information, an abnormality of a mounting state of a sensor to a subject, and
an abnormality of a measurement environment of the physiological information.

15. A non-transitory computer-readable medium configured to store a program for causing a computer to execute tire-following-step:
calculate an alarm generation time, in which at least one of a vital alarm notifying an abnormality of physiological information measured by a sensor and a technical alarm notifying an abnormality related to measurement of the physiological information is generated, or a ratio of the alarm generation time; and
output the calculated alarm generation time or the calculated ratio.
